(19) United States
(12) Patent Application Publication
(10) Pub. No.: US 2024/0398844 A1
(43) Pub. Date: Dec. 5, 2024

(54) PHENYLCOUMARINS FOR TREATING INFLAMMATION

(71) Applicants: Gerardo David ANAYA EUGENIO, Columbus, OH (US); Rachel MATA, Mexico City (MX); Isabel RIVERO CRUZ, Mexico City (MX); Ohio State Innovation Foundation, Columbus, OH (US)

(72) Inventors: Esperanza CARCACHE DE BLANCO, Columbus, OH (US); Nicole A. WOODARD, Philadelphia, PA (US); Gerardo David ANAYA EUGENIO, Columbus, OH (US); Rachel MATA, Mexico City (MX); Isabel RIVERO CRUZ, Mexico City (MX)

(21) Appl. No.: 18/696,044

(22) PCT Filed: Sep. 29, 2022
(86) PCT No.: PCT/US2022/045242

§ 371 Date: Mar. 27, 2024

Related U.S. Application Data
(60) Provisional application No. 63/249,879, filed on Sep. 29, 2021.
(60) Provisional application No. 63/254,266, filed on Oct. 11, 2021.

(51) Int. Cl. A61K 31/7048; C07H 17/075

(52) U.S. Cl. CPC A61K 31/7048; C07H 17/075

(57) ABSTRACT

The present disclosure is directed to compounds of Formula I, compositions, and methods of using the same in the treatment or prevention of inflammation or disorders or conditions associated with the same comprising administering a therapeutically effective amount of a compound described herein, or a pharmaceutically acceptable salt, prodrug, or derivative thereof.

### CROSS-REFERENCE TO RELATED APPLICATIONS

This application claims the benefit of priority to U.S. Provisional Application No. 63/249,879, filed Sep. 29, 2021, and U.S. Provisional Application No. 63/254,266, filed Oct. 11, 2021, each disclosure of which is incorporated herein by reference in its entirety.

### TECHNICAL FIELD

This disclosure relates to compounds, compositions, and their use in treating inflammation in subjects in need thereof.

### BACKGROUND

Inflammation is a complex biological process that occurs in response to harmful stimuli, such as for example infections, damage to cells or tissue, or irritants. While inflammation is vital for healing and combating infection, abnormal or excessive inflammation can adversely affect the health, comfort and/or mobility of a subject.

A wide range of anti-inflammatory agents are known, including steroids (such as glucocorticoids) and non-steroidal anti-inflammatory drugs (such as aspirin, ibuprofen, and naproxen). However, these drugs may be ineffective at treating some inflammatory conditions or may be associated with adverse side effects.

For example, some current anti-inflammatory agents have adverse side effects including gastrointestinal damage, renal damage, photosensitivity, hepatic stimulation, headaches, dizziness, Cushing's syndrome, hypertension, hypokalemia, or hypernatremia. Further, some anti-inflammatory agents may not be suitable for certain subjects, such as pregnant subjects or those with inflammatory bowel disease, due to these potential adverse effects.

Thus, there is a clear need for the development of new methods for treating or preventing inflammation, as well as any disorders or conditions associated with inflammation.

### SUMMARY

The present disclosure provides compounds of Formula I and their use in methods for the treatment of inflammation and disorders associated thereof.

Thus, in one aspect, a compound of Formula I is provided:

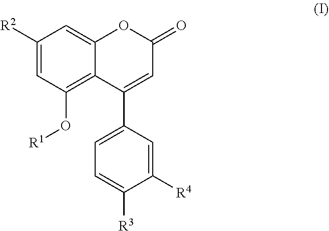

or a pharmaceutically acceptable salt, prodrug, or derivative thereof;

wherein all variables are as further defined herein.

In another aspect, a method is provided for the treatment or prevention of inflammation in a subject in need thereof comprising administering a therapeutically effective amount of a compound described herein, or a pharmaceutically acceptable salt, prodrug, or derivative thereof.

In a further aspect, a method is provided for the treatment of one or more symptoms resulting from inflammation in a subject in need thereof comprising administering a therapeutically effective amount of a compound described herein, or a pharmaceutically acceptable salt, prodrug, or derivative thereof.

In yet another aspect, a method is provided for the treatment of an inflammatory disorder in a subject in need thereof comprising administering a therapeutically effective amount of a compound described herein, or a pharmaceutically acceptable salt, prodrug, or derivative thereof.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 depicts type 2 diabetic zebrafish expression (from top to bottom) of β-actin, HbA1C, PPAR-γ, and PEPCK in treatment groups: A) healthy, untreated; B) diabetic, untreated; C) diabetic, 10 μM rosiglitazone, D) diabetic, 10 μM compounds 16 through 39 from the diverse library.

FIG. 2 depicts Western blot band density for HbA1C expression in zebrafish treatment groups: A) healthy, untreated; B) diabetic, untreated; C) diabetic, 10 μM rosiglitazone; D) diabetic, 10 μM compounds 16 through 39 from diverse library.

FIG. 3 depicts Western blot band density for PPAR-γ expression in zebrafish treatment groups: A) healthy, untreated; B) diabetic, untreated; C) diabetic, 10 μM rosiglitazone; D) diabetic, 10 μM compounds 16 through 39 from diverse library.

FIG. 4 depicts Western blot density for PEPCK expression in zebrafish treatment groups: A) healthy, untreated; B) diabetic, untreated; C) diabetic, 10 μM rosiglitazone; D) diabetic, 10 μM compounds 16 through 39 from diverse library.

FIG. 5 is Western blot analysis of zebrafish treatment groups A) healthy, untreated; B) diabetic, untreated; C) diabetic, rosiglitazone; D) diabetic, 10 μM compounds 22 through 24 from diverse library.

FIG. 6 is duplicate Western blot analysis of zebrafish treatment groups: 1) healthy, untreated; 2) diabetic, untreated; 3) diabetic, 10 μM rosiglitazone; 4) diabetic, 10 μM 24; 5) diabetic, 5 μM 24; 6) diabetic, 1 μM 24; 7) diabetic, 10 μM 40; 8) diabetic, 5 μM 40; 9) diabetic, 1 μM 40.

FIG. 7 is Western blot band densities for HbA1C expression in zebrafish treatment groups: 1) healthy, untreated; 2) diabetic, untreated; 3) diabetic, 10 μM rosiglitazone; 4) diabetic, 10 μM 24; 5) diabetic, 5 μM 24; 6) diabetic, 1 μM 24; 7) diabetic, 10 μM 40; 8) diabetic, 5 μM 40; 9) diabetic, 1 μM 40.

FIG. 8 is Western blot band densities for PEPCK expression in zebrafish treatment groups: 1) healthy, untreated; 2) diabetic, untreated; 3) diabetic, 10 μM rosiglitazone; 4) diabetic, 10 μM 24; 5) diabetic, 5 μM 24; 6) diabetic, 1 μM 24; 7) diabetic, 10 μM 40; 8) diabetic, 5 μM 40; 9) diabetic, 1 μM 40.

FIG. 9 is Western blot band densities for PPAR-γ expression in zebrafish treatment groups: 1) healthy, untreated; 2) diabetic, untreated; 3) diabetic, 10 μM rosiglitazone; 4) diabetic, 10 μM 24; 5) diabetic, 5 μM 24; 6) diabetic, 1 μM 24; 7) diabetic, 10 μM 40; 8) diabetic, 5 μM 40; 9) diabetic, 1 μM 40.

FIG. 10 is Western blot band densities for NFκB p65 expression in zebrafish treatment groups: 1) healthy, untreated; 2) diabetic, untreated; 3) diabetic, 10 μM rosiglitazone; 4) diabetic, 10 μM 24; 5) diabetic, 5 μM 24; 6) diabetic, 1 μM 24; 7) diabetic, 10 μM 40; 8) diabetic, 5 μM 40; 9) diabetic, 1 μM 40; p=0.0101.

FIG. 11 is Western blot band density in larval diabetic zebrafish treated with 10 μM rosiglitazone (+) or 4-phenylcoumarin glycosides 22, 23, or 24 from the diverse library.

FIG. 12 depicts Zebrafish treated with cycloheximide, compound 24, and compound 40 as a control for evaluation of compound toxicity.

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain, benefiting from the teachings presented in the descriptions herein and the associated drawings. Therefore, it is understood that the disclosures are not limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual embodiments described and illustrated herein has discrete components and features that may be readily separated from or combined with the features of any of the other several embodiments without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present invention is not entitled to antedate such publication by prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

### Definitions

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise. Thus, for example, reference to “a compound,” “a composition,” or “a disorder” includes, but is not limited to, two or more such compounds, compositions, or disorders, and the like.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x,’ ‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term “therapeutically effective amount” refers to an amount sufficient to achieve the desired therapeutic result or to have an effect on undesired symptoms but generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the particular compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to permanently halt the progression of the disease. This can be monitored by routine diagnostic methods known to one of ordinary skill in the art for any particular disease. The desired response to treatment of the disease or condition can also be delaying the onset or even preventing the onset.

For example, it is well within the skill of the art to start doses of a compound at levels lower than those required to achieve the desired therapeutic effect and to increase the dosage gradually until the desired effect is achieved. If desired, the effective daily dose can be divided into multiple doses for administration. Consequently, single dose compositions can contain such amounts or submultiples thereof to make up the daily dose. The individual physician can adjust the dosage in the event of any contraindications. It is generally preferred that a maximum dose of the pharmacological agents of the invention (alone or in combination with other therapeutic agents) be used, that is, the highest safe dose according to sound medical judgment. However, a patient may insist on a lower or tolerable dose for medical reasons, psychological reasons, or virtually any other reason.

A response to a therapeutically effective dose of a disclosed compound or composition can be measured by determining the physiological effects of the treatment or medication, such as the decrease or lack of disease symptoms following the administration of the treatment or pharmacological agent. Other assays will be known to one of ordinary skill in the art and can be employed for measuring the level of the response. The amount of a treatment may be varied, for example, by increasing or decreasing the amount of a disclosed compound or pharmaceutical composition, changing the disclosed compound or pharmaceutical composition administered, changing the route of administration, changing the dosage timing, and so on. Dosage can vary and can be administered in one or more dose administrations daily for one or several days. Guidance can be found in the literature for appropriate dosages for given classes of pharmaceutical products.

As used herein, “prevent” or “preventing” refers to precluding, averting, obviating, forestalling, stopping, or hindering something from happening, especially by advance action. Where reduce, inhibit or prevent are used herein, unless specifically indicated otherwise, the use of the other two words is also expressly disclosed.

As used herein, “optional” or “optionally” means that the subsequently described event or circumstance can or cannot occur. The description includes instances where said event or circumstance occurs and those where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, “treating” and “treatment” generally refer to obtaining a desired pharmacological or physiological effect. The effect can be but does not necessarily have to be prophylactic in preventing or partially preventing a disease, symptom, or condition such as inflammation. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress; and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound or a pharmaceutical composition thereof calculated to produce the desired response or responses in association with its administration.

As used herein, “therapeutic” can refer to treating, healing, or ameliorating a disease, disorder, condition, or side effect or decreasing the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some embodiments from 1 to 3 or from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one embodiment, the total number of S and O atoms in the heteroaryl ring is not more than 2. In another embodiment, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC-(CH₂)₁₋₄-COOH, and the like, or using a different acid that produced the same counterion. Lists of additional suitable salts may be found, e.g., in Remington's Pharmaceutical Sciences, 17^{h} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds

The present disclosure provides compounds of Formula I which are useful in the treatment or prevention of inflammation in a subject in need thereof.

In one aspect, a compound of Formula I is provided:

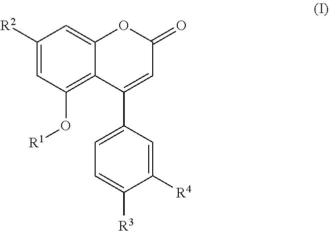

or a pharmaceutically acceptable salt, prodrug, or derivative thereof;

wherein:
R¹ is

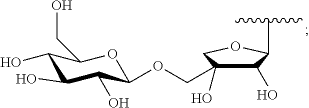

R², R³, and R⁴ are each independently selected from hydrogen, halo, cyano, azido, C₁-C₆alkyl, C₁-C₆haloalkyl, R^{x}O-, and R^{x}R^{y}N-, wherein R³ and R⁴ cannot both hydroxyl;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some embodiments, R² is selected from halogen, C₁-C₆ alkyl, and R^{x}O-. In some embodiments, R³ is selected from halogen, C₁-C₆ alkyl, and R^{x}O-. In some embodiments, R⁴ is selected from halogen, C₁-C₆ alkyl, and R^{x}O-.

In some embodiments, R² is R^{x}O-. In some embodiments, R³ is R^{x}O-. In some embodiments, R⁴ is R^{x}O-. In some embodiments, R² is halogen (for example, fluorine, chlorine, bromine, or iodine). In some embodiments, R² is C₁-C₆ alkyl (for example, methyl). In some embodiments, R³ is halogen (for example, fluorine, chlorine, bromine, or iodine). In some embodiments, R³ is C₁-C₆ alkyl (for example, methyl). In some embodiments, R⁴ is halogen (for example, fluorine, chlorine, bromine, or iodine). In some embodiments, R⁴ is C₁-C₆ alkyl (for example, methyl).

In some embodiments, R^{x} is selected from C₁-C₆ alkyl. In some embodiments, R^{x} is selected from methyl, ethyl, n-propyl, and isopropyl.

In some embodiments, R² is methoxy. In some embodiments, R³ is alkoxy (e.g., isopropoxy) and R⁴ is hydroxy. In some embodiments, R³ is hydroxy and R⁴ is alkoxy (e.g., isopropoxy).

In some embodiments, the compound of Formula I is

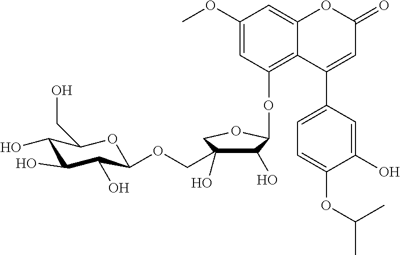

or a pharmaceutically acceptable salt, prodrug, or derivative thereof.

The present disclosure also includes compounds of Formula I or Formula II with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (3H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d₆-acetone, or d₆-DMSO. A solvate can be in a liquid or solid form.

A “prodrug” as used herein means a compound which when administered to a host in vivo is converted into a parent drug. As used herein, the term “parent drug” means the presently described compound herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner.

For example, non-limiting embodiments include the use of a divalent linker moiety such as a dicarboxylic acid, amino acid, diamine, hydroxycarboxylic acid, hydroxyamine, dihydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a hydroxyl group on the parent drug and a carboxylic acid on the prodrug moiety to form an ester; a hydroxyl on the parent drug and a hydroxylated prodrug moiety to form an ester; a hydroxyl on the parent drug and a phosphonate on the prodrug to form a phosphonate ester; a hydroxyl on the parent drug and a phosphoric acid prodrug moiety to form a phosphate ester; a hydroxyl on the parent drug and a prodrug of the structure HO−(CH₂)₂-O-(C₂₋₂₄ alkyl) to form an ether; a hydroxyl on the parent drug and a prodrug of the structure HO—(CH₂)₂—S—(C₂₋₂₄ alkyl) to form an thioether; and a hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of inflammation in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80, buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, camauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary and can be administered in one or more dose administrations daily, for one or several days.

### Methods of Treatment

In one aspect, a method is provided for the treatment or prevention of inflammation in a subject in need thereof comprising administering a therapeutically effective amount of a compound described herein, or a pharmaceutically acceptable salt, prodrug, or derivative thereof.

In another aspect, a method is provided for the treatment of one or more symptoms resulting from inflammation in a subject in need thereof comprising administering a therapeutically effective amount of a compound described herein, or a pharmaceutically acceptable salt, prodrug, or derivative thereof.

In yet another aspect, a method is provided for the treatment of an inflammatory disorder in a subject in need thereof comprising administering a therapeutically effective amount of a compound described herein, or a pharmaceutically acceptable salt, prodrug, or derivative thereof.

In another aspect, a method is provided for the treatment or prevention of inflammation in a subject in need thereof comprising administering a therapeutically effective amount of a compound of Formula II

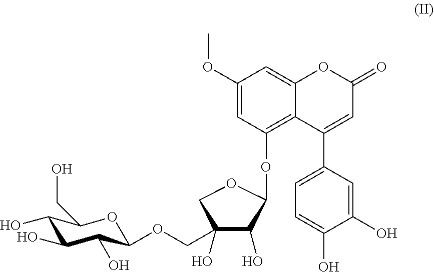

or a pharmaceutically acceptable salt, prodrug, or derivative thereof.

In another aspect, a method is provided for the treatment of one or more symptoms resulting from inflammation in a subject in need thereof comprising administering a therapeutically effective amount of a compound of Formula II, or a pharmaceutically acceptable salt, prodrug, or derivative thereof.

In yet another aspect, a method is provided for the treatment of an inflammatory disorder in a subject in need thereof comprising administering a therapeutically effective amount of a compound of Formula II, or a pharmaceutically acceptable salt, prodrug, or derivative thereof.

In some embodiments, the methods described herein can be used to treat inflammation caused by: a physical cause such as burns, frostbite, physical injury (either blunt or penetrating), foreign bodies (including splinters, dirt, or debris), trauma, or ionizing radiation; a biological cause such as infection by a pathogen, an immune reaction due to hypersensitivity, or stress; or a chemical cause such as a chemical irritant, a toxin.

In some embodiments, the inflammation comprises acute inflammation. In some embodiments, the acute inflammation may be in response to one or more of the following: a wound (such as a cut, bruise, or burn); an infection (such as a bacterial, viral, fungal, or protist infection); exposure to a toxin or ionizing radiation; exposure to an allergen or antigen; and the presence of a foreign body (for example, a splinter) in a subject.

In some embodiments, the inflammation comprises chronic inflammation. In some embodiments, the chronic inflammation may be associated with a persistent form of acute inflammation, as described above, or may be associated with an inflammatory disorder.

The present methods may be used to treat or prevent inflammation in any part of the body, including but not limited to inflammation of: the central nervous system (such as encephalitis, myelitis, or meningitis); the peripheral nervous system (such as neuritis); the eye (such as dacryoadenitis, scleritis, episcleritis, or keratitis); the ear (such as otitis); the heart (such as endocarditis, myocarditis, or pericarditis); the vascular system (such as arteritis, phlebitis, or capillaritis); the respiratory system (such as sinusitis, rhinitis, pharyngitis, epiglottitis, laryngitis, tracheitis, bronchitis, pneumonitis, or pleurisy); the digestive system (such as stomatitis, gingivitis, glossitis, tonsillitis, sialadenitis, parotitis, cheilitis, pulpitis, gnathitis, oesophagitis, gastritis, gastroenteritis, enteritis, colitis, pancolitis, appendicitis, cryptitis, hepatitis, cholecystitis, or pancreatitis); the integumentary system (such as dermatitis or mastitis); the musculoskeletal system (such as arthritis, myositis, synovitis, tenosynovitis, or bursitis); the urinary system (such as nephritis, ureteritis, cystitis, or urethritis); the female reproductive system (such as oophoritis, salpingitis, endometritis, myometritis, parametritis, cervicitis, vaginitis, or vulvitis); the male reproductive system (such as orchitis, epididymitis, prostatitis, vasculitis, balanitis, or posthitis); the endocrine system (such as insulitis, hypophysitis, thyroiditis, parathyroiditis, or adrenalitis); or the lymphatic system (such a lymphangitis or lymphadenitis).

The present methods may also be used to treat or prevent inflammation resulting from an inflammatory disorder. In some embodiments, the methods described herein may be used as an analgesic to treat pain, for example a headache. In some embodiments, the methods described herein may be used to treat arthritis, including but not limited to rheumatoid arthritis, spondyloarthopathies, gouty arthritis, systemic lupus erythematosus, osteoarthritis, and juvenile arthritis. In some embodiments, the methods described herein may be used to treat asthma, bronchitis, menstrual cramps, tendinitis, bursitis, and skin related conditions such as psoriasis, eczema, burns and dermatitis. In some embodiments, the methods described herein may be used to treat gastrointestinal conditions such as inflammatory bowel disease, Crohn's disease, gastritis, irritable bowel syndrome, and ulcerative colitis. In some embodiments, the methods described herein may be used to treat inflammation present in a disorder including, but not limited to, vascular disease, migraine headaches, perarteritis nodosa, thyroiditis, aplastic anemia, Hodgkin's disease, scleroderma, rheumatic fever, type I diabetes, myasthenia gravis, sarcoidosis, nephrotic syndrome, Behcet's syndrome, polymyositis, hypersensitivity, conjunctivitis, gingivitis, swelling occurring after an injury, myocardial ischemia, and the like.

In some embodiments, the methods described herein may be used to treat or prevent inflammation associated with a disorder including, but not limited to, acne vulgaris, asthma, an autoimmune disease, an autoinflammatory disease, celiac disease, chronic prostatitis, colitis, diverticulitis, glomerulonephritis, hidradenitis suppurativa, hypersensitivities, inflammatory bowel disease, interstitial cystitis, lichen planus, mast cell activation syndrome, otitis, pelvic inflammatory disease, reperfusion injury, rheumatic fever, rheumatoid arthritis, rhinitis, sarcoidosis, transplant rejection, or vasculitis. In some embodiments, the methods described herein may be used to treat or prevent inflammation associated with atherosclerosis, cancer, or ischemic heart disease.

In some embodiments, the methods described herein may be used to treat a systemic inflammatory disorder or ameliorate or diminish one or more inflammatory symptoms of a system inflammatory disorder including, but not limited to, non-alcoholic fatty liver disease, non-alcoholic steatohepatitis, inflammatory bowel disease, Crohn's disease, ulcerative colitis, psoriasis, irritable bowel syndrome, ankylosing spondylitis, osteoporosis, rheumatoid arthritis, psoriatic arthritis, chronic obstructive pulmonary disease, atherosclerosis, pulmonary arterial hypertension, pyridoxine-dependent epilepsy, atopic dermatitis, rosacea, multiple sclerosis, systemic lupus erythematosus, lupus nephritis, sepsis, eosinophilic esophagitis, chronic kidney disease, fibrotic renal disease, chronic eosinophilic pneumonia, extrinsic allergic alveolitis, pre-eclampsia, endometriosis, polycystic ovary syndrome, or cyclophosphamide-induced hemorrhagic cystitis.

In some embodiments, the methods described herein may be used to treat inflammation resulting from a disorder selected from light chain deposition disease, IgA nephropathy, end-stage renal disease, gout, pseudogout, diabetic nephropathy, diabetic neuropathy, traumatic brain injury, noise-induced hearing loss, Alzheimer's disease, Parkinson's disease, Huntington disease, amyotrophic lateral sclerosis, primary biliary cirrhosis, primary sclerosing cholangitis, uterine leiomyoma, sarcoidosis, or chronic kidney disease.

Further embodiments of the disclosure include:

Embodiment 1. A compound of Formula I:

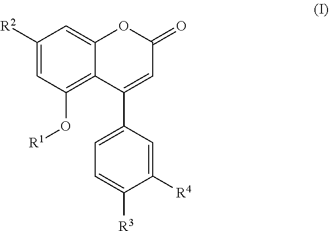

or a pharmaceutically acceptable salt, prodrug, or derivative thereof;

wherein:
R¹ is

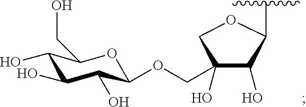

R², R³, and R⁴ are each independently selected from hydrogen, halo, cyano, azido, C₁-C₆alkyl, C₁-C₆haloalkyl, R^{x}O-, and R^{x}R^{y}N-, wherein R³ and R⁴ cannot both hydroxyl;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.
   Embodiment 2. The compound of embodiment 1, wherein R² is selected from halogen, C₁-C₆ alkyl, and R^{x}O-.
   Embodiment 3. The compound of embodiment 1 or embodiments 2, wherein R² is R^{x}O-, wherein R^{x} is C₁-C₆ alkyl.
   Embodiment 4. The compound of any one of embodiments 1-3, wherein R² is R^{x}O-, wherein R² is selected from methyl, ethyl, n-propyl, and isopropyl.
   Embodiment 5. The compound of any one of embodiments 1-4, wherein R² is methoxy.
   Embodiment 6. The compound of any one of embodiments 1-5, wherein R³ is selected from halogen, C₁-C₆ alkyl, and R^{x}O-.
   Embodiment 7. The compound of any one of embodiments 1-6, wherein R³ is Rb-, wherein R^{x} is hydrogen or C₁-C₆ alkyl.
   Embodiment 8. The compound of any one of embodiments 1-7, wherein R⁴ is selected from halogen, C₁-C₆ alkyl, and R^{x}O-.
   Embodiment 9. The compound of any one of embodiments 1-8, wherein R⁴ is R^{x}O-, wherein R^{x} is hydrogen or C₁-C₆ alkyl.
   Embodiment 10. The compound of any one of embodiments 1-4, wherein R³ is alkoxy and R⁴ is hydroxy.
   Embodiment 11. The compound of embodiment 10, wherein R³ is isopropoxy.
   Embodiment 12. The compound of any one of embodiments 1-4, wherein R³ is hydroxy and R⁴ is alkoxy.
   Embodiment 13. The compound of embodiment 12, wherein R⁴ is isopropoxy.
   Embodiment 14. The compound of any one of embodiments 1-4, wherein R³ and R⁴ are each alkoxy.
   Embodiment 15. The compound of embodiment 14, wherein R³ and R⁴ are each isopropoxy.
   Embodiment 16. A pharmaceutical composition comprising a compound of any one of embodiments 1-15, or a pharmaceutically acceptable salt, prodrug, or derivative thereof, and a pharmaceutically acceptable carrier or excipient.
   Embodiment 17. A method for treating inflammation in a subject in need thereof comprising administering a compound of any one of embodiments 1-15, or a pharmaceutically acceptable salt, prodrug, or derivative thereof, or a pharmaceutical composition of embodiment 16.
   Embodiment 18. A method for diminishing or ameliorating one or more symptoms caused by inflammation in a subject in need thereof comprising administering a compound of any one of embodiments 1-15, or a pharmaceutically acceptable salt, prodrug, or derivative thereof, or a pharmaceutical composition of embodiment 16.
   Embodiment 19. A method for preventing inflammation in a subject in need thereof comprising administering a compound of any one of embodiments 1-15, or a pharmaceutically acceptable salt, prodrug, or derivative thereof, or a pharmaceutical composition of embodiment 16.
   Embodiment 20. The method of any one of embodiments 17-19, wherein the inflammation is acute inflammation.
   Embodiment 21. The method of any one of embodiments 17-19, wherein the inflammation is chronic inflammation.
   Embodiment 22. The method of any one of embodiments 17-19, wherein the inflammation is associated with an inflammatory disorder.
   Embodiment 23. The method of any one of embodiments 17-19, wherein the inflammation is selected from encephalitis, myelitis, meningitis, neuritis, dacryoadenitis, scleritis, episcleritis, keratitis, otitis, endocarditis, myocarditis, pericarditis, arteritis, phlebitis, capillaritis, sinusitis, rhinitis, pharyngitis, epiglottitis, laryngitis, tracheitis, bronchitis, pneumonitis, pleurisy, stomatitis, gingivitis, glossitis, tonsillitis, sialadenitis, parotitis, cheilitis, pulpitis, gnathitis, oesophagitis, gastritis, gastroenteritis, enteritis, colitis, pancolitis, appendicitis, cryptitis, hepatitis, cholecystitis, pancreatitis, dermatitis, mastitis, arthritis, myositis, synovitis, tenosynovitis, bursitis, nephritis, ureteritis, cystitis, urethritis, oophoritis, salpingitis, endometritis, myometritis, parametritis, cervicitis, vaginitis, vulvitis, orchitis, epididymitis, prostatitis, vasculitis, balanitis, posthitis, insulitis, hypophysitis, thyroiditis, parathyroiditis, adrenalitis, lymphangitis and lymphadenitis.
   Embodiment 24. The method of any one of embodiments 17-19, wherein the inflammation is associated with arthritis.
   Embodiment 25. The method of embodiment 24, wherein the arthritis is selected from rheumatoid arthritis, spondyloarthopathies, gouty arthritis, systemic lupus erythematosus, osteoarthritis, and juvenile arthritis.
   Embodiment 26. The method of any one of embodiments 17-19, wherein the inflammation is associated with asthma, bronchitis, menstrual cramps, tendinitis, bursitis, psoriasis, eczema, burns or dermatitis.
   Embodiment 27. The method of any one of embodiments 17-19, wherein the inflammation is associated with a gastrointestinal condition.
   Embodiment 28. The method of embodiment 27, wherein the gastrointestinal condition is selected from inflammatory bowel disease, Crohn's disease, gastritis, irritable bowel syndrome, and ulcerative colitis.
   Embodiment 29. The method of any one of embodiments 17-19, wherein the inflammation is associated with vascular disease, migraine headaches, perarteritis nodosa, thyroiditis, aplastic anemia, Hodgkin's disease, scleroderma, rheumatic fever, type I diabetes, myasthenia gravis, sarcoidosis, nephrotic syndrome, Behcet's syndrome, polymyositis, hypersensitivity, conjunctivitis, gingivitis, swelling occurring after an injury, or myocardial ischemia.
   Embodiment 30. A method for the treatment of a systemic inflammatory disorder in a subject in need thereof comprising administering a compound of any one of embodiments 1-15, or a pharmaceutically acceptable salt, prodrug, or derivative thereof, or a pharmaceutical composition of embodiment 16.
   Embodiment 31. A method for ameliorating or diminishing one or more inflammatory symptoms of a systemic inflammatory disorder in a subject in need thereof comprising administering a compound of any one of embodiments 1-15, or a pharmaceutically acceptable salt, prodrug, or derivative thereof, or a pharmaceutical composition of embodiment 16.
   Embodiment 32. The method of any one of embodiments 30 or 31, wherein the systemic inflammatory disorder is selected from non-alcoholic fatty liver disease, non-alcoholic steatohepatitis, inflammatory bowel disease, Crohn's disease, ulcerative colitis, psoriasis, irritable bowel syndrome, ankylosing spondylitis, osteoporosis, rheumatoid arthritis, psoriatic arthritis, chronic obstructive pulmonary disease, atherosclerosis, pulmonary arterial hypertension, pyridoxine-dependent epilepsy, atopic dermatitis, rosacea, multiple sclerosis, systemic lupus erythematosus, lupus nephritis, sepsis, eosinophilic esophagitis, chronic kidney disease, fibrotic renal disease, chronic eosinophilic pneumonia, extrinsic allergic alveolitis, pre-eclampsia, endometriosis, polycystic ovary syndrome, and cyclophosphamide-induced hemorrhagic cystitis.
   Embodiment 33. A method for treating inflammation in a subject in need thereof comprising administering a therapeutically effective amount of a compound of Formula II

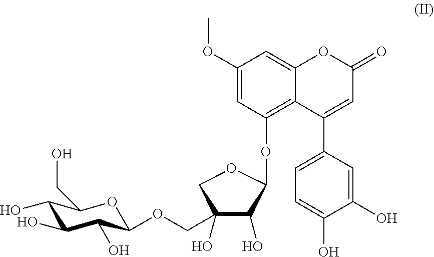

or a pharmaceutically acceptable salt, prodrug, or derivative thereof.
Embodiment 34. A method for diminishing or ameliorating one or more symptoms caused by inflammation in a subject in need thereof comprising administering a therapeutically effective amount of a compound of Formula II

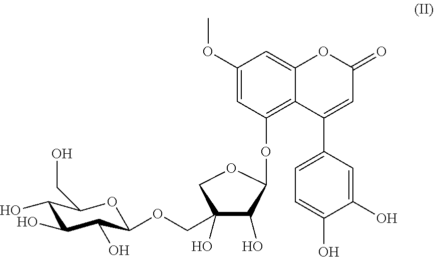

or a pharmaceutically acceptable salt, prodrug, or derivative thereof.
Embodiment 35. A method for preventing inflammation in a subject in need thereof comprising administering a therapeutically effective amount of a compound of Formula II

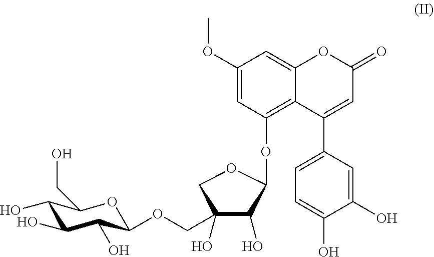

or a pharmaceutically acceptable salt, prodrug, or derivative thereof.
Embodiment 36. The method of any one of embodiments 33-35, wherein the inflammation is acute inflammation.
Embodiment 37. The method of any one of embodiments 33-35, wherein the inflammation is chronic inflammation.
Embodiment 38. The method of any one of embodiments 33-35, wherein the inflammation is associated with an inflammatory disorder.
Embodiment 39. The method of any one of embodiments 33-35, wherein the inflammation is selected from encephalitis, myelitis, meningitis, neuritis, dacryoadenitis, scleritis, episcleritis, keratitis, otitis, endocarditis, myocarditis, pericarditis, arteritis, phlebitis, capillaritis, sinusitis, rhinitis, pharyngitis, epiglottitis, laryngitis, tracheitis, bronchitis, pneumonitis, pleurisy, stomatitis, gingivitis, glossitis, tonsillitis, sialadenitis, parotitis, cheilitis, pulpitis, gnathitis, oesophagitis, gastritis, gastroenteritis, enteritis, colitis, pancolitis, appendicitis, cryptitis, hepatitis, cholecystitis, pancreatitis, dermatitis, mastitis, arthritis, myositis, synovitis, tenosynovitis, bursitis, nephritis, ureteritis, cystitis, urethritis, oophoritis, salpingitis, endometritis, myometritis, parametritis, cervicitis, vaginitis, vulvitis, orchitis, epididymitis, prostatitis, vasculitis, balanitis, posthitis, insulitis, hypophysitis, thyroiditis, parathyroiditis, adrenalitis, lymphangitis and lymphadenitis.
Embodiment 40. The method of any one of embodiments 33-35, wherein the inflammation is associated with arthritis.
Embodiment 41. The method of embodiment 40, wherein the arthritis is selected from rheumatoid arthritis, spondyloarthopathies, gouty arthritis, systemic lupus erythematosus, osteoarthritis, and juvenile arthritis.
Embodiment 42. The method of any one of embodiments 33-35, wherein the inflammation is associated with asthma, bronchitis, menstrual cramps, tendinitis, bursitis, psoriasis, eczema, burns or dermatitis.
Embodiment 43. The method of any one of embodiments 33-35, wherein the inflammation is associated with a gastrointestinal condition.
Embodiment 44. The method of embodiment 43, wherein the gastrointestinal condition is selected from inflammatory bowel disease, Crohn's disease, gastritis, irritable bowel syndrome, and ulcerative colitis.
Embodiment 45. The method of any one of embodiments 33-35, wherein the inflammation is associated with vascular disease, migraine headaches, perarteritis nodosa, thyroiditis, aplastic anemia, Hodgkin's disease, scleroderma, rheumatic fever, type I diabetes, myasthenia gravis, sarcoidosis, nephrotic syndrome, Behcet's syndrome, polymyositis, hypersensitivity, conjunctivitis, gingivitis, swelling occurring after an injury, or myocardial ischemia.
Embodiment 46. A method for the treatment of a systemic inflammatory disorder in a subject in need thereof comprising administering a therapeutically effective amount of a compound of Formula II

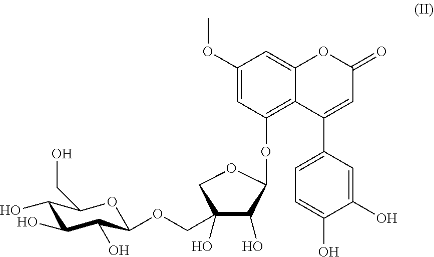

or a pharmaceutically acceptable salt, prodrug, or derivative thereof.
Embodiment 47. A method for ameliorating or diminishing one or more inflammatory symptoms of a systemic inflammatory disorder in a subject in need thereof comprising administering a therapeutically effective amount of a compound of Formula II

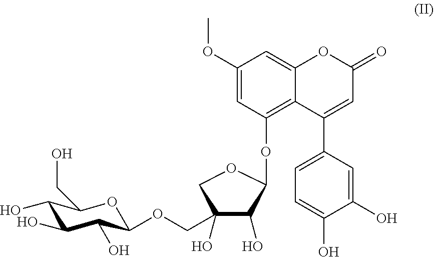

or a pharmaceutically acceptable salt, prodrug, or derivative thereof.
Embodiment 48. The method of any one of embodiments 46 or 47, wherein the systemic inflammatory disorder is selected from non-alcoholic fatty liver disease, non-alcoholic steatohepatitis, inflammatory bowel disease, Crohn's disease, ulcerative colitis, psoriasis, irritable bowel syndrome, ankylosing spondylitis, osteoporosis, rheumatoid arthritis, psoriatic arthritis, chronic obstructive pulmonary disease, atherosclerosis, pulmonary arterial hypertension, pyridoxine-dependent epilepsy, atopic dermatitis, rosacea, multiple sclerosis, systemic lupus erythematosus, lupus nephritis, sepsis, eosinophilic esophagitis, chronic kidney disease, fibrotic renal disease, chronic eosinophilic pneumonia, extrinsic allergic alveolitis, pre-eclampsia, endometriosis, polycystic ovary syndrome, and cyclophosphamide-induced hemorrhagic cystitis.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are put forth to provide those of ordinary skill in the art with a complete disclosure and description of how the compounds, compositions, and methods claimed herein are made and evaluated and are intended to be purely exemplary of the invention and are not intended to limit the scope of what the inventors regard as their invention. Efforts have been made to ensure accuracy concerning numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Western Blot Procedure

Alloxan-induced diabetic zebrafish were treated with compounds for immunoblot to validate the newly developed animal model, determine the antidiabetic potential of natural product leads, evaluate glucose metabolism, and discover possible mechanisms of action.

At 72 hpf, naturally dechorionated zebrafish were sorted into a 24-well microplate with 10 fish per well. Type 2 diabetes was induced with the diabetogenic agent alloxan at a dose of 0.5 mg/mL in system water for 3 hours. The incubation solution was changed to 0.01% DMSO in system water with compounds at concentrations of 20 μg/mL or 10 PM, respectively. After 3 hours of treatment with natural product samples, 10 mg/mL D-glucose was administered in system water for 30 minutes, followed by 1 hour in fresh system water to metabolize the glucose and natural product leads. Upon completion of the 4-phase treatment cycle, zebrafish treatment groups were snap frozen into individual microcentrifuge tubes on dry ice, stored overnight at −80° C., thawed, homogenized with 2 μL PBS per animal, and centrifuged at 4° C. for 1 hour at 17,000 rpm. Protein content of homogenate supernatant was determined using the Pierce BCA Protein Assay kit (Pierce Biotechnology) and bovine serum albumin (BSA) standard curve. Absorbance of protein solutions from zebrafish homogenates and serial dilutions of the BSA standard were measured at 544 nm, using a FLUOstar Optima plate reader (BMG Labtechnologies GmbH, Inc.). Homogenates were analyzed by Western blot. Equal amounts of protein (20 μg) were combined with 4× lithium dodecyl sulphate (LDS) loading buffer (NuPAGE, Life Technologies, Carlsbad, CA, USA), brought to volume (15 μL) with PBS, and heated to 95° C. for 5 minutes. Proteins were resolved using NuPAGE Bis-Tris 4-12% gradient sodium dodecyl sulfate polyacrylamide gel electrophoresis (SDS-PAGE) gels together with SeeBlue Plus2 Pre-Stained Standard Ladder (Life Technologies).

Electrophoresis was performed using NuPAGE MES SDS Running Buffer and XCell SureLock Mini-Cell Electrophoresis System (Life Technologies). Separated proteins were transferred from the gel to a polyvinylidene fluoride (PVDF) membrane using tris buffered saline with Tween-20 (TBST) buffer. The blots were blocked in 3% BSA in TBST and subsequently probed using primary antibodies (1:1000 in 1% BSA in TBST) against each target overnight (Table 1). The secondary, horseradish peroxidase (HRP) conjugated antibodies (1:2000) were obtained from Santa Cruz Biotechnology Inc., (Santa Cruz, CA, USA). Conjugated antibodies were detected using SuperSignal West Femto chemiluminescent substrate from Pierce Biotechnology. Western blots were visualized, and images captured using a luminometer and LumiAnalyst 3.0 software (Boehringer-Mannheim, Mannheim, Germany). Band density was quantified for proteins of interest using ImageJ Gel Analysis.

In the case of duplicate blots, the band density results were expressed in bar graphs as the mean±standard error of the mean (SEM) of two independent experiments. Due to the labor- and time-intensive aspect of Western blot analysis with multiple target proteins, a maximum of two independent immunoblot experiments were performed per compared sample set. However, due to each treatment group having been a pooled sample of 10 individual zebrafish, it was decided that the statistical reliability was sufficient for identifying promising lead compounds for further investigation. The statistical analysis of the Western blot experiments was performed using GraphPad Prism 8 (GraphPad Software, San Diego, CA, USA). Statistical analysis of the data obtained was made by one-way analysis of variance (ANOVA), followed by a Dunnett's post hoc test, in comparison to the diabetic, untreated group of zebrafish with p values included in the figure captions. Additionally, treatment groups were also evaluated versus the diabetic, untreated control group for significance by Student's f-test and indicated in the bar graphs with p values represented by *p<0.05, **p<0.01, and ***p<0.001.

TABLE 1
Antibody manufacturers and molecular weights

| Antibody | Supplier | kDa |
|---|---|---|
| GLUT-4 | Santa Cruz Biotechnology, Inc., Dallas, TX, USA | 50-63 |
| (IF8) | | |
| HbA_{1C} | MyBioSource, Inc., San Diego, CA, USA | 65-68 |
| IRAP | Santa Cruz Biotechnology, Inc., Dallas, TX, USA | 140 |
| NFκB p65 | Cell Signaling Technology, Inc., Danvers, MA, USA | 65 |
| PEPCK | Santa Cruz Biotechnology, Inc., Dallas, TX, USA | 62 |
| (F-3) | | |
| PPAR-γ | Proteintech Group, Inc., Rosemont, IL, USA | 58 |
| TNKS | Aviva Systems Biology Corporation, San Diego, CA, | 144 |
| | USA | |
| B-actin | Santa Cruz Biotechnology, Inc., Dallas, TX, USA | 43 |
| (N-21) | | |

### Zebrafish Western Blot Analysis of Diverse Library

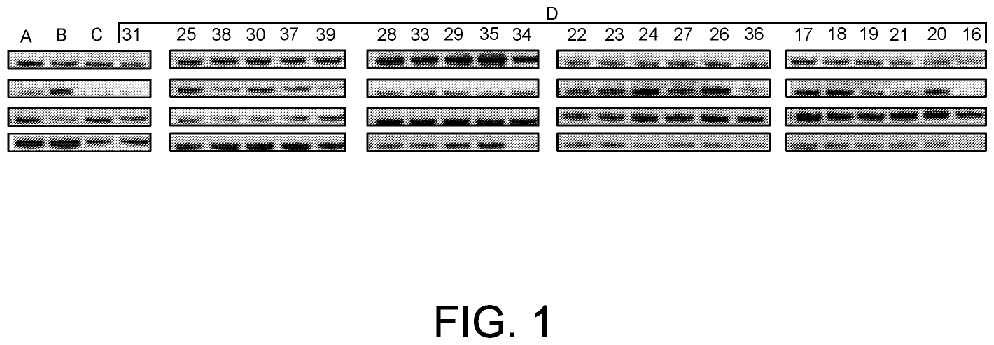

A diverse library of natural product compounds (see below) was screened in the diabetic zebrafish Western blot assay. Each compound 16 through 39 was tested at 10 μM in phase 2 of a 4-phase treatment cycle as detailed above. Western blot procedure with results displayed in FIG. 1. Compound 32 was toxic to all 10 fish at 10 μM for 3 hours, excluding it from level of expression data. Expression of glycated hemoglobin HbA1C, thiazolidinedione target PPAR-γ, and gluconeogenic enzyme PEPCK were analyzed to determine the compounds with the best lead compounds in terms of hypoglycemic activity.

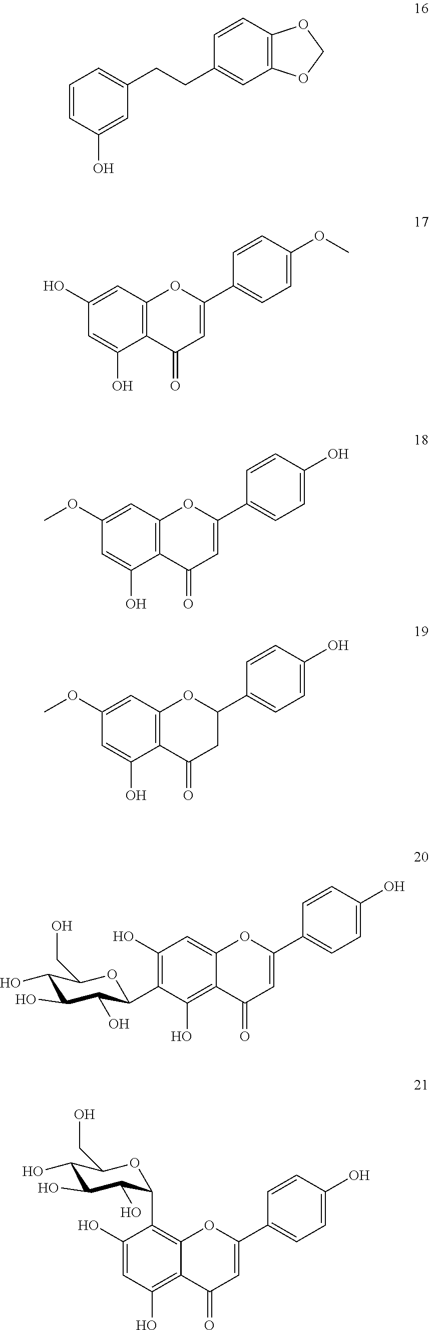

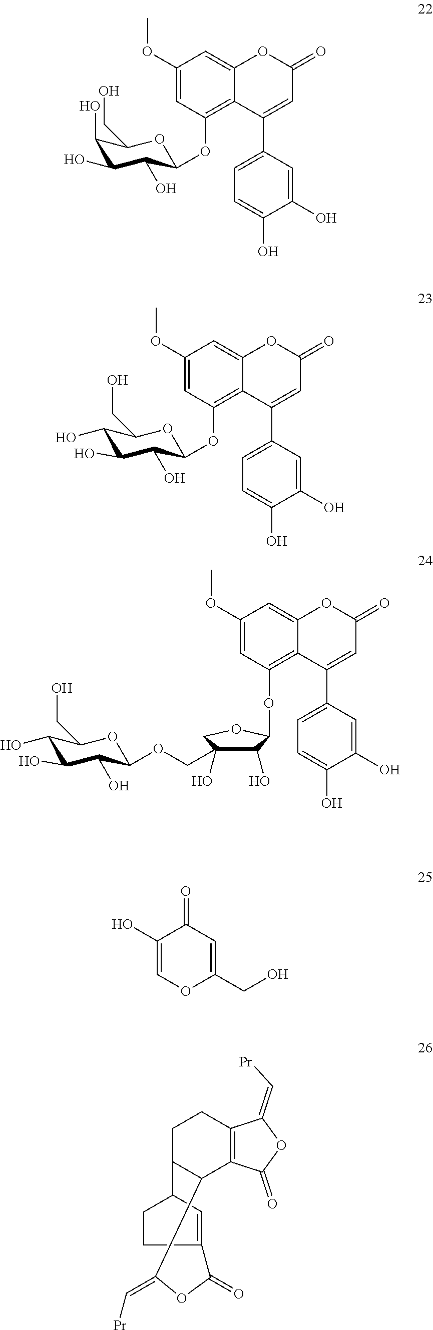

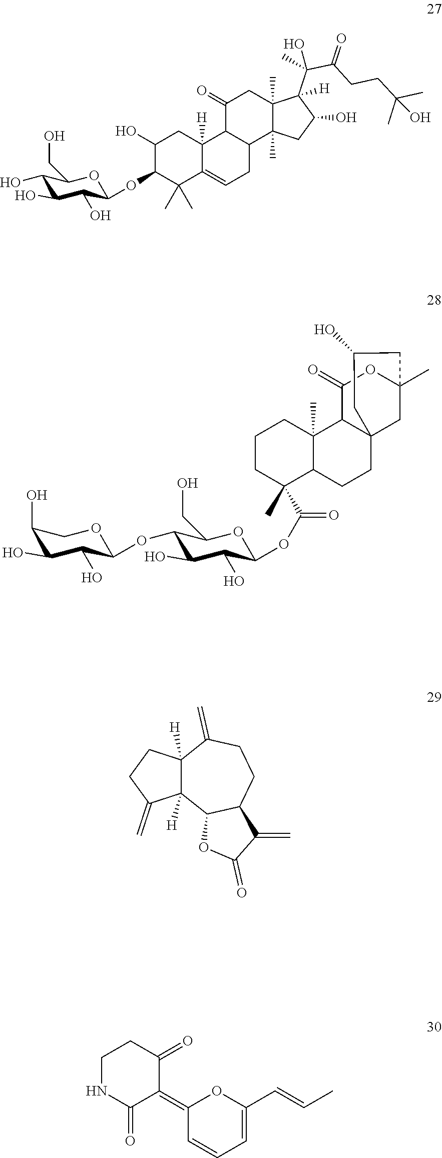

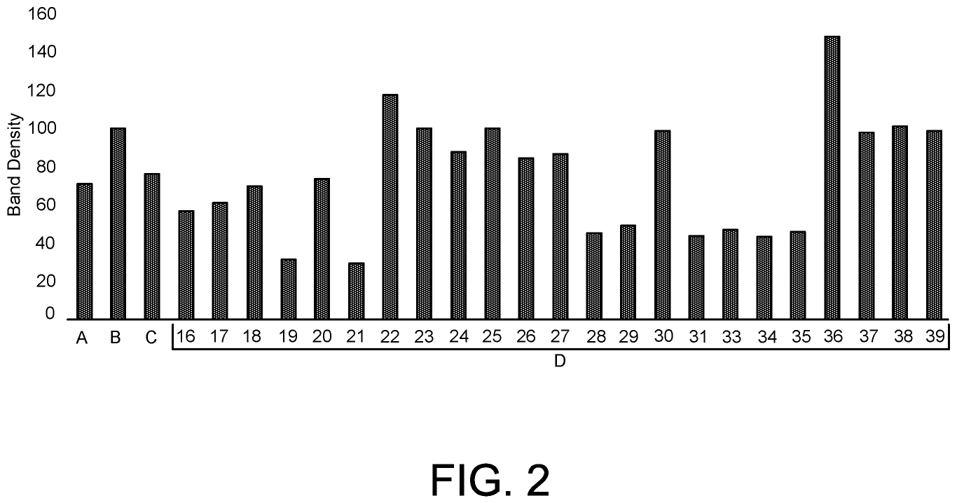

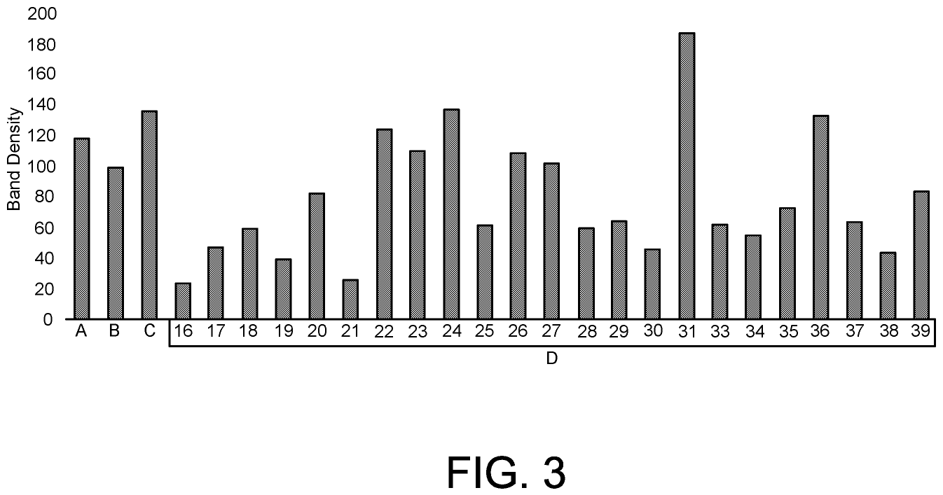

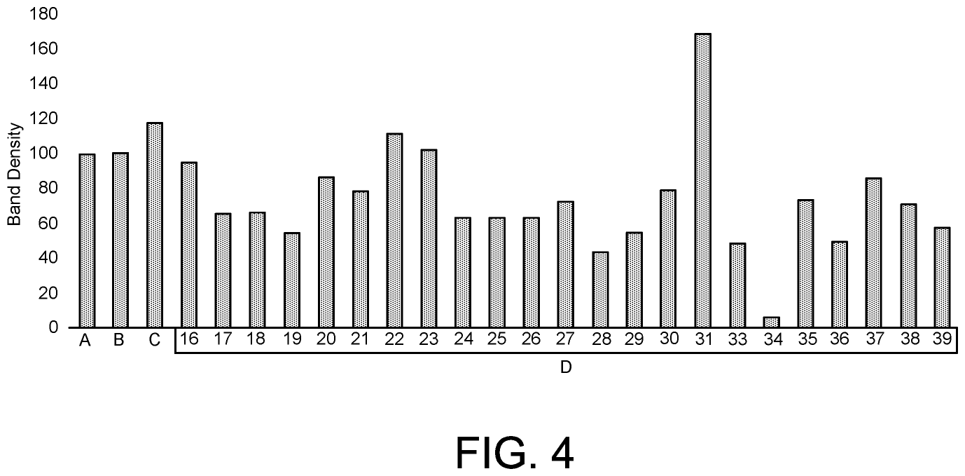

To analyze the Western blot data, band density was determined using ImageJ software and displayed in bar graphs for HbA1C (FIG. 2), PPAR-γ (FIG. 3), and PEPCK (FIG. 4) expression. Band density was calculated in comparison to β-actin and normalized to the untreated diabetic group of zebrafish.

### Bioactivity of Compounds in Diverse Library

In addition to the Western blot, the diverse library was also screened in the in vitro PPAR-γ assay. Samples from the diverse library of natural product compounds were tested in the PPAR-γ in vitro assay to prioritize the best lead compounds for potential antidiabetic activity. The compounds were tested at 50 μg/mL according to the protocol detailed in section 3.2.3. PPAR-γ ELISA Assay in HeLa cells, with results displayed in (Table 2).

TABLE 2
PPAR-γ activity of diverse library compounds (50 μg/mL)

| Compound | % Activation PPAR-γ |
|---|---|
| 16 | 0 |
| 17 | 0 |
| 18 | 0 |
| 19 | 77 |
| 20 | 89 |
| 21 | 57 |
| 22 | 21 |
| 23 | 145 |
| 24 | 101 |
| 25 | 53 |
| 26 | 0 |
| 27 | 65 |
| 28 | 0 |
| 29 | 0 |
| 30 | 37 |
| 31 | 153 |
| 32 | NT |
| 33 | NT |
| 34 | 277 |
| 35 | 1 |
| 36 | 49 |
| 37 | 0 |
| 38 | 0 |
| 39 | NT |
| Rosiglitazone | 100 |

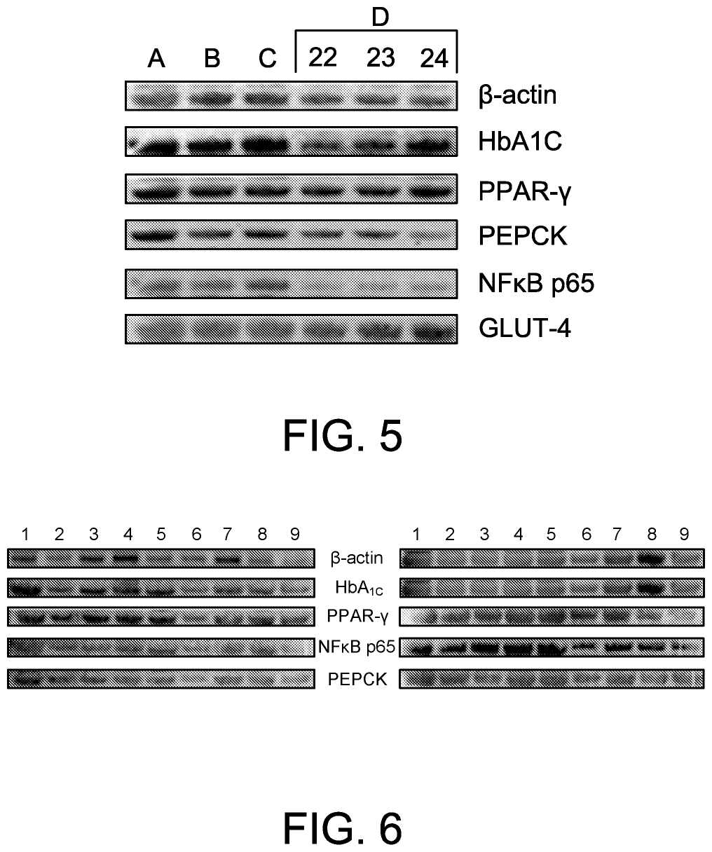

Based on the zebrafish Western blot data and in vitro PPAR-Y activity, a series of 4-phenylcoumarin glycosides from the diverse library were selected for further investigation. Expression of NFκB and GLUT-4 were also included in the Western blot analysis for compounds 22, 23, and 24 (FIG. 5).

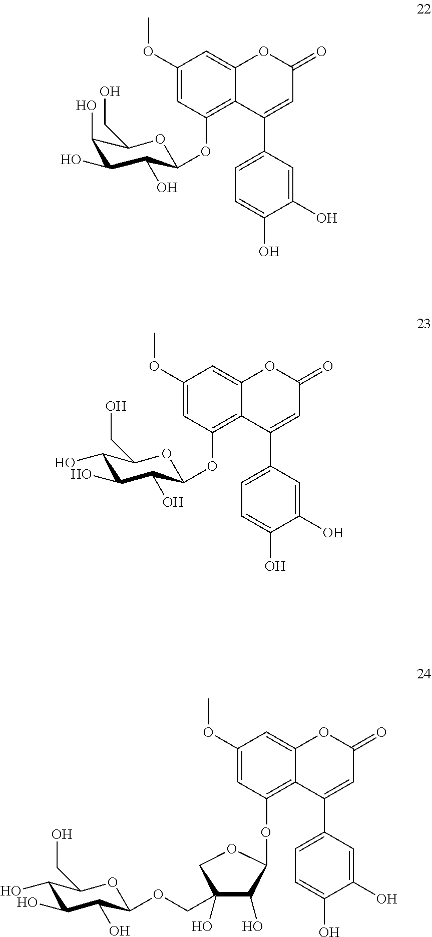

Due to the quantity available and its interesting sugar moiety, compound 24 was selected for derivatization in an effort to improve its potential antidiabetic activity and probe possible mechanisms of action.

Derivatization of a Plant Natural Product from Diverse Library

The library of plant and fungal natural product compounds were screened in the panel of in vitro bioassays and diabetic zebrafish model. Due to its bioactivity, 5-O-[β-D-apiofuranosyl-(1→6)-β-D-glucopyranosyl]-7-methoxy-3′,4′-dihydroxy-4-phenylcoumarin (24) was selected as a promising lead and derivatized to explore improvement of its potential as an antidiabetic drug lead.

### Derivatization Reaction

A scintillation vial was charged with 24 (27 mg, 0.0454 mmol, 1 equiv.), followed by potassium carbonate (31.38 mg, 0.227 mmol, 5 equiv.) and methanol (500 μL). 2-Bromopropane (12.80 μL, 0.1362 mmol, 3 equiv.) was then charged to the vial and heated to 70° C. for 12 h. After cooling to room temperature, the reaction mixture was filtered through a syringe filter (0.45 μm), concentrated to dryness, and purified via reversed-phase HPLC.

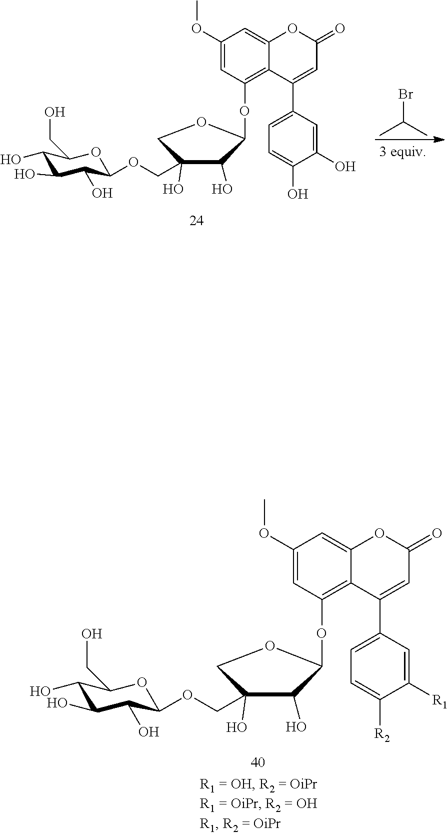

### HPLC Purification of Derivative 40

The isopropyl etherification reaction products were separated by reversed-phase HPLC using a Waters 600 controller pump and 996 photodiode array detector (Waters, Milford, MA, USA) and a Varian Polaris C18-A (21.2×250 mm, 10 μm) preparative column from Agilent Technologies (Santa Clara, CA, USA). The isolation was pursued using an isocratic binary mobile phase of 7:3 methanol to water with 7.0 mL/min flow rate. The reaction products were dissolved in methanol to a concentration of approximately 25 mg/mL and 100 μL serially injected. Four fractions were obtained over serial 12 minute runs, with the retention time and yields reported in Table 3. Fractions 1 and 3 were evaluated for purity by 1H NMR spectroscopy, and fraction 3 was later identified as an isopropyl ether derivative (40) of the parent 4-phenylcoumarin glycoside 24.

TABLE 3
Reversed-phase HPLC of the isopropyl
etherification reaction products

| | Initial Retention Time | Fraction Retention Time | Yield |
|---|---|---|---|
| Fraction | (min) | (min) | (mg) |
| 1 | 5.6 | 6.1 | 1.5 |
| 2 | 6.6 | ND | 0.9 |
| 3 | 8.6 | 9.3 | 7.2 |
| 4 | 9.4 | ND | ND |

### Characterization of Derivative

Bruker Avance NEO 400 MHz spectrometer (Bruker Scientific LLC, Billerica, MA, USA) was used to record ¹H and ¹³C NMR data at 300 K using standard Bruker pulse sequences. 1H NMR spectra were recorded at 400 MHz with tetramethylsilane (TMS) as an internal standard. Derivative fractions 1 and 3 from the HPLC separation were evaluated for purity by ¹H NMR and compared to the parent compound (24). Fraction 3 appeared to be semi-pure with the targeted derivative (40) as the main component.

Solubility of the fractions was a challenge, and chloroform-d (CDCl₃), pyridine45, methanol-d₄ (CD₃OD), dimethyl sulfoxide-d₆, and water-d₂ (D₂O) (Cambridge Isotope Laboratories, Tewksbury, MA, USA) were all tried as solvents. Fraction 1 was most soluble in CDCl₃; however, there were several different signals indicative of isopropyl groups present, suggesting a mixture of structurally similar compounds. Since there was only 1.5 mg of material, further purification and structure elucidation were not pursued.

Fraction 3 was most soluble in D₂O, and at approximately 11 mg/mL, ¹H and ¹³C NMR data were obtained in an effort to confirm the presence of an isopropyl ether derivative (40). However, solubility of fraction 3 continued to be problematic even in D₂O. Further acquisition attempts with increased probe temperature (310 K) were no more successful at keeping the compound (40) in solution. The original isolation and elucidation of the parent compound (24) was reported in 2005 using CD₃OD as the NMR solvent, but with a high isolation yield (1.2 g of 24), solubility issues to the extent of obscuring NMR data were not encountered. Additionally, 40 is even less soluble than 24, so the ¹H NMR spectrum of 40 in CD₃OD did not contain any peaks distinguishable from noise except water and the solvent peak.

In a final effort to overcome the solubility issue, fraction 3 (derivative 40) was for access to a stronger instrument, the 700 MHz Bruker Avance III HD Ascend NMR (Bruker Scientific LLC). A ¹H NMR spectrum of the derivative (40, approximately 10 mg/mL in CDCb) with increased resolution was obtained; unfortunately, contamination of the sample with water, starting material, and/or other unknown contaminants also increased due to repeated drying and resuspension in various NMR solvents.

Despite insolubility and impurities, overlaying the CDCl₃ spectra of 24 (400 MHz, 1.2 mg/mL) and 40 (700 MHz, 10 mg/mL) highlighted the appearance of a doublet corresponding to the geminal dimethyl protons on the isopropoxy group in 40 at SH 0.918. However, due to contamination at 6H 3.49, integration ratios between the methoxy protons and the isopropoxy geminal dimethyl groups could not be completed to determine whether the derivative was mono- or di-isopropylated. Additionally, the abundance of signals from the disaccharide moiety between dx 3.0-5.0 prohibited distinction between the presence of one or two protons splitting into septets, corresponding to the methine proton of the mono- or di-isopropylated ether. Without a complete NMR dataset, structure elucidation was impossible and only key comparisons could be made. For more substantial evidence of having formed and purified derivative 40, the parent compound (24) and supposed derivative (fraction 3) were analyzed by mass spectrometry (MS).

Mass spectrometry of 24 and its derivative 40 were performed. Molecular weights were obtained with a 15T Bruker SolariX XR Fourier-transform ion cyclotron resonance mass spectrometer (Bruker Scientific LLC) using high resolution electrospray ionization in the positive-ion mode.

According to the MS spectrum of 24, the most abundant ion present had m/z 617.14750, corresponding to the [M+Na]⁺ peak of a molecule with chemical formula C₂₇H₃₀O₁₅ (that of 24). The calculated [M+Na]⁺ for C₂₇H₃₀O₁₅ (m/z 617.147691) yielded a mass error of 0.3 ppm, an acceptable mass error (<0.5 ppm) for high resolution electrospray ionization mass spectrometry (HRESIMS). In addition, the [M+H]⁺ and [M+K]⁺ ions were also identified in the spectrum.

With molecular weight 636.21, the mono-isopropoxy derivative 40 was identified in fraction 3 by the [M+K]⁺ peak at m/z 675.26108. The mass error exceeded 0.5 ppm, which may have been due to the challenging solubility of 40. A small amount of the di-isopropoxy ether derivative was also identified by the [M+H]⁺ peak at m/z 679.43914, most likely corresponding to the right-hand shoulder of the HPLC peak representing fraction 3. Due to resonance, the most nucleophilic oxygen would be para; therefore, if only one site were to be alkylated, it would have preferentially occurred at the para oxygen. Thus, the derivative was proposed to have the structure 5-O-[β-D-apiofuranosyl-(1→6)-β-D-glucopyranosyl]-7-methoxy-3′,4′-dihydroxy-4-phenylcoumarin. To provide additional evidence of having formed and isolated 40 from the isopropyl etherification reaction products, 24 and 40 were also characterized by specific rotation. Further characterization efforts are ongoing.

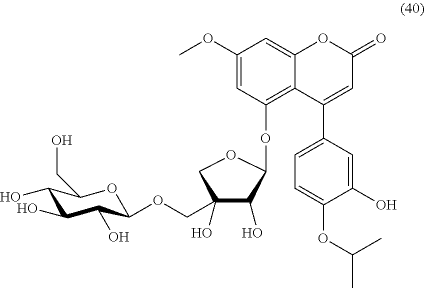

Specific rotation of 4-phenylcoumarin glycoside 24 and derivative 40 was determined using the Anton Paar MCP 150 Modular Compact polarimeter (Anton Paar USA Inc., Ashland, VA, USA). The compounds 24 and 40 were characterized and identified by comparing their physical properties and spectroscopic data with literature reports and to one another.

### Characterization of 4-Phenylcoumarin Glycoside 24

Brown crystals; [α]²⁰D −52.0 (c 0.05, MeOH); HRESIMS observed m/z 617.14750 [M+Na]⁺ (calculated for C₂₇H₃₀O₁₅Na, 617.147691).

### Characterization of Isopropyl Ether Derivative 40

Tan amorphous solid; [α]²⁰D +43.0 (c 0.1, MeOH). ¹H NMR key signals: SH 0.918 [d, OiPr (CH₃)₂]; HRESIMS observed m/z 675.26108 [M+K]⁺ (calculated for C₃₀H₃₆O₁₅K, 675.168579).

### Bioactivity of 4-Phenylcoumarin Glycosides and Derivative 40

Following derivatization and purification, compound 40 was tested in the NFκB and PPAR-Y in vitro assays, as well as the diabetic zebrafish Western blot. The three 4-phenylcoumarin glycoside natural products from the diverse library (22, 23, and 24) and the isopropyl ether derivative (40), were evaluated for PPAR-γ activity at 50 μg/mL in HeLa cells according to a PPAR-γ ELISA Assay (Table 4). Compounds 24 and 40 were also tested in the NFκB in vitro assay (at 50, 5, 0.5, and 0.05 μM) to determine their IC50 values for inhibiting NFκB translocation to the nucleus, to determine their NFκB IC50 following the protocol detailed in section 3.2.2. NFκB Assay (Table 4).

TABLE 4
NFκB and PPAR-γ data of diverse library 4-
phenylcoumarin glycosides and derivative 40

| | | |
|---|---|---|
| Compound | NFκB IC₅₀ | % Activation PPAR-γ |

| 22 | NT | | 21 |
|---|---|---|---|
| 23 | NT | | 145 |
| 24 | 3.95 | μM | 101 |
| 40 | 10.23 | μM | 0 |

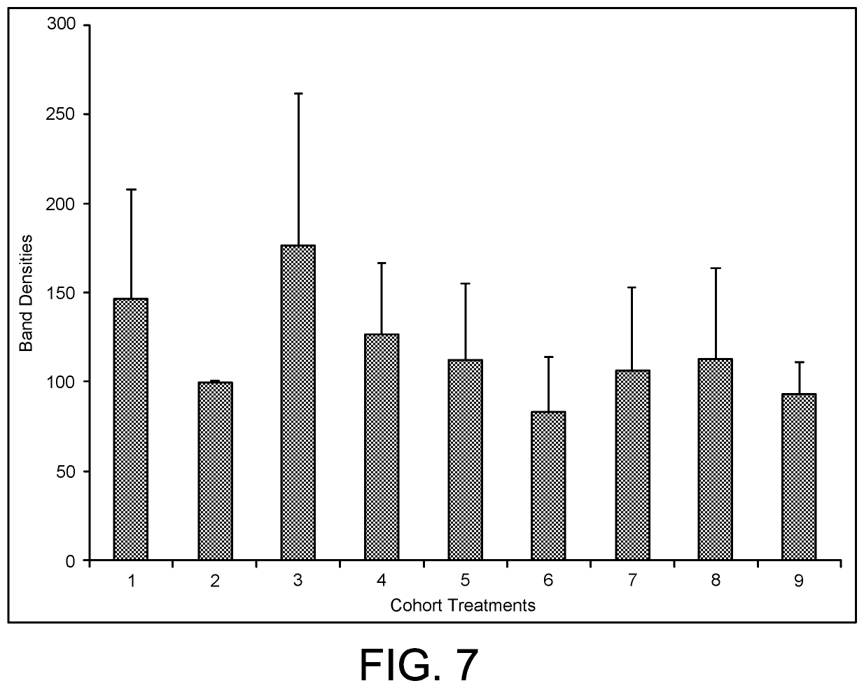

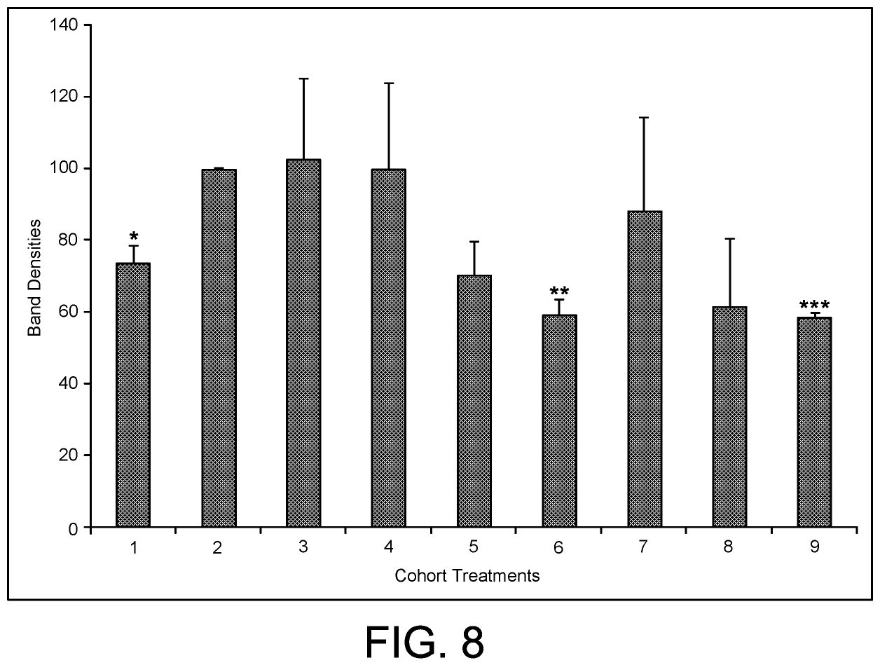

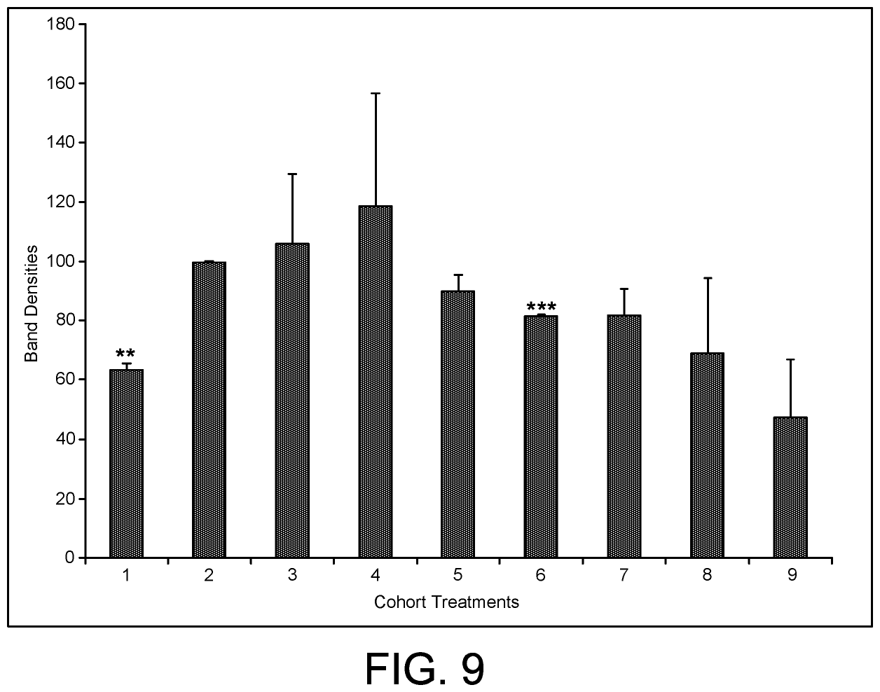

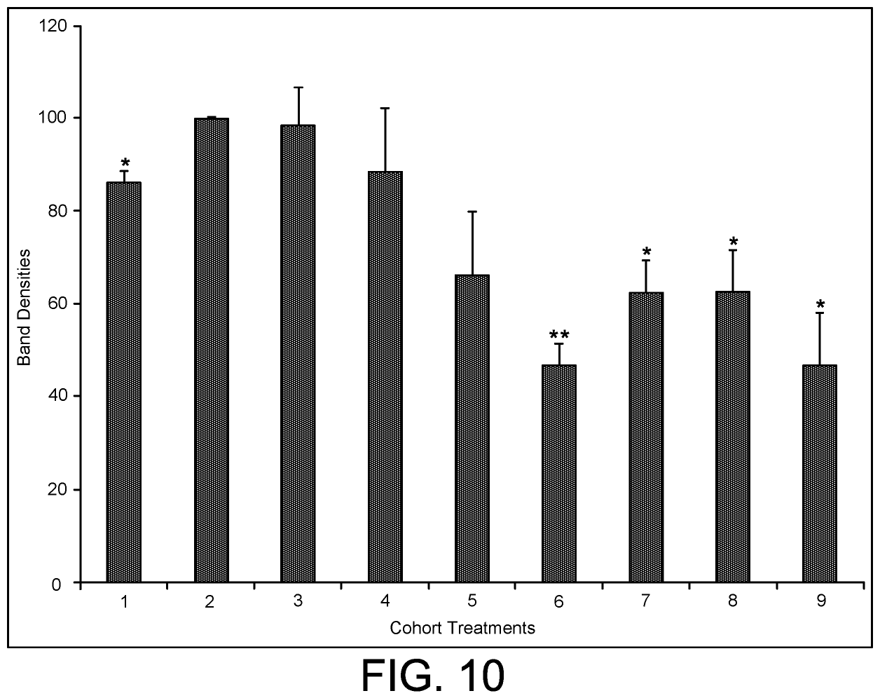

Expression levels of molecular targets, glycated hemoglobin, and metabolic enzymes were evaluated in diabetic zebrafish treated with compound 24 and its derivative (40). Both compounds were tested at concentrations of 10, 5, and 1 μM in fish water with 0.01% DMSO, according to the 4-phase treatment cycle described above. Duplicate blots were run with treatment groups of 10 zebrafish each. The developed blots were imaged (FIG. 6), and band densities were determined through ImageJ analysis and represented in bar graphs for expression of HbA1C (FIG. 7), PEPCK (FIG. 8), PPAR-γ (FIG. 9), and NFκB (FIG. 10).

### Results and Discussion

### Bioactivity of Diverse Library

The library of diverse natural product compounds consisted of 24 compounds that had previously demonstrated some hypoglycemic activity in streptozotocin diabetic rats. To probe possible mechanisms of action for their hypoglycemic effects and to validate the type 2 diabetic zebrafish model developed in the current example, the library compounds were screened in the in vitro PPAR-γ assay and the zebrafish immunoblot.

Ten of the compounds showed greater PPAR-γ activity in the in vitro assay than the positive control rosiglitazone, when each of the samples was tested at 50 μg/mL in HeLa cells (Table 2). The four compounds that exhibited the greatest PPAR-γ agonism were 34>31>23>24, with 2.7- to 7.5-fold greater specific binding than rosiglitazone. According to the PPAR-γ expression of zebrafish in the immunoblot screening of the diverse library (FIG. 1 with PPAR-7 band densities represented in FIG. 3), compounds 31 and 24 exhibited greater PPAR-7 expression than the PPAR-γ positive control, rosiglitazone, followed closely by compounds 36, 22, and 23. The in vitro and in vivo data therefore share compounds 31, 24, and 23 as top performers, instilling confidence in those small molecules as strong leads in the antidiabetic drug discovery program Importantly, compounds 22-24 share the structural skeleton of 4-phenylcoumarin glycosides and may offer an opportunity for brief structure-activity relationship insight.

As shown in FIG. 2, the members of the diverse library that exhibited the lowest glycated hemoglobin levels were 21 and 19, followed by a cluster of compounds (34<31<28<35<33<29). There was a total of 13 natural products in the library screening that caused a greater reduction of HbA1C expression than the thiazolidinedione, rosiglitazone, which was also significantly reduced in comparison to untreated diabetic zebrafish, as expected. Therefore, in terms of hypoglycemic activity without discriminating a particular mechanism of action, compounds 21 and 19 showed the greatest antidiabetic potential, and compound 31 should be included as a high performer in both HbA1C reduction and PPAR-γ agonism.

Based on expression of gluconeogenic enzyme PEPCK (FIG. 4), the library compounds with the greatest hypoglycemic effects were 31>rosiglitazone>22>23. The remaining members of the library show equal or lesser PEPCK expression to both non-diabetic and untreated diabetic zebrafish, indicating the zebrafish remain hyperglycemic. The only compound that may represent a risk of reaching hypoglycemia, according to the results in FIG. 4 and the fact that rosiglitazone is known to exert hypoglycemic effects without risk of clinical hypoglycemia, is compound 31 due to its exceptionally strong PEPCK expression. However, that does not preclude compound 31 from having great antidiabetic potential but is a point of concern for future studies.

Considering the in vitro PPAR-γ activity and in vivo PPAR-γ, HbA1C, and PEPCK expression, compounds 22-24 and 31 showed the greatest antidiabetic potential, and because of their structural similarity and amount of material, compounds 22-24 were selected as the first candidates for further investigation.

### Bioactivity of Three 4-Phenylcoumarin Glycosides and a Derivative

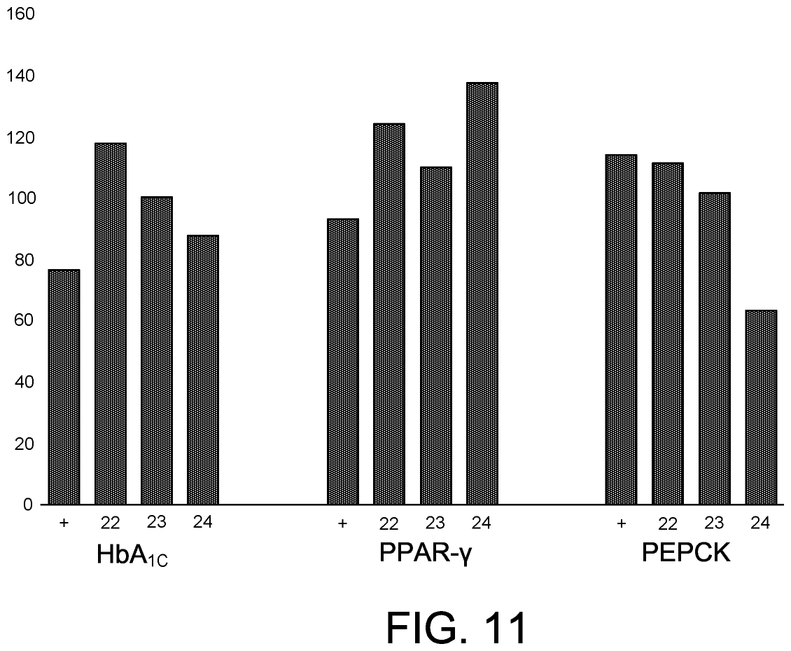

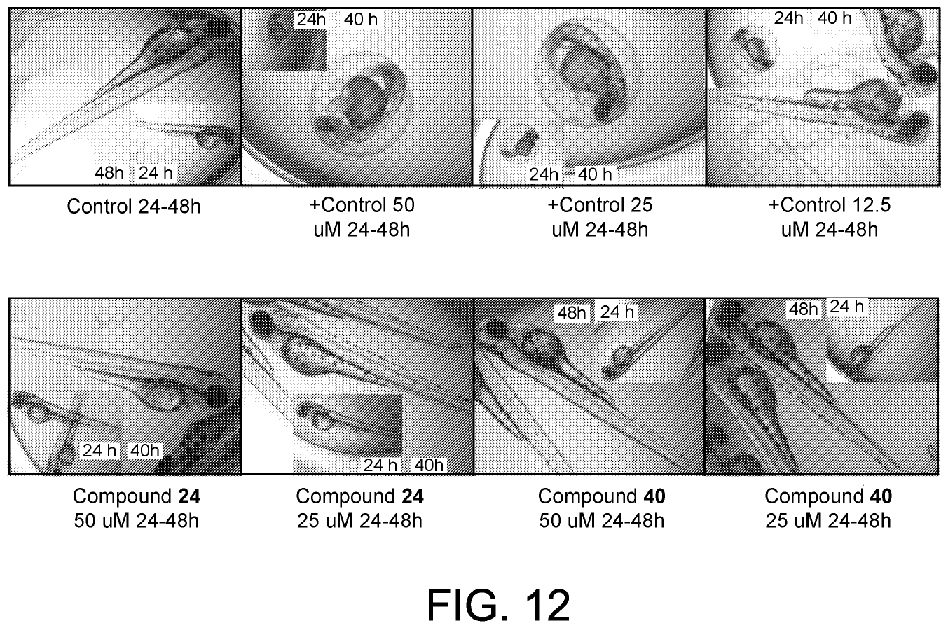

To summarize the diverse library screening Western blot data that pertains only to the 4-phenylcoumarin glycosides (22, 23, and 24), an additional bar graph has been organized by protein (FIG. 11). Of the three library compounds that share a structural skeleton, 23 and 24 were top performers in the in vitro PPAR-γ assay (Table 2); and in the larval diabetic zebrafish blots, 24 was the most effective in reducing glycated hemoglobin, 24 exhibited the greatest PPAR-γ activation surpassing even the positive control thiazolidinedione, and 22 exerted the greatest PEPCK expression (FIG. 11). Additionally, all three compounds exhibited excellent inhibition of NFκB expression and strong GLUT-4 expression (FIG. 5).

As an excellent lead, 24 was derivatized to yield 40, and both compounds were evaluated in the NFκB and PPAR-γ in vitro assays (Table 4). The derivative (40) was 2.6-times less effective at inhibiting NFκB translocation in HeLa cells than its parent compound 24, and in the PPAR-γ ELISA, the isopropyl etherification caused a loss of all activity (Table 4), which may or may not be related to its insolubility. To investigate 24 and 40 in a live, metabolizing system with appropriate hormone responses, the compounds were tested (at 10, 5, and 1 μM) in the larval diabetic zebrafish assay.

### HbA_{1C} Bioactivity of 24 and 40 in Diabetic Zebrafish

Through Western blot analysis, HbA1C band densities for diabetic zebrafish treated with 24 exhibited reduced glycated hemoglobin levels in comparison to rosiglitazone (FIG. 6 and FIG. 7). However, the HbA1C expression of rosiglitazone-treated and untreated diabetic zebrafish was opposite of the hypoglycemic effect expected. The dose-dependent effect of 24 also opposite of the expected results, but taking error bars into consideration, it is possible that there was no dose-dependent effect. Since the positive control, an FDA-approved drug known to exert a hypoglycemic effect, was administered at 10 μM, it is highly probable that 10, 5, and 1 μM of 24 were too dilute to significantly reduce HbA1C and in a dose-dependent manner. Derivative 40, administered at the same dosages, was also lacking a dose-dependent response. In comparing expression levels between 24 and 40, neither compound was significantly more effective than the other. To understand the HbA1C results, additional replicates and higher doses of 24 and 40 are necessary.

### PEPCK Bioactivity of 24 and 40 in Diabetic Zebrafish

Although the HbA1C data left more questions than answers, the PEPCK expression of diabetic zebrafish treated with 24 and its derivative 40 was more meaningful. Compound 24 exhibited a dose-dependent increase in PEPCK expression, with the highest concentration (10 μM) having a greater gluconeogenic effect than rosiglitazone (10 μM), indicating a return to normoglycemia after the induced hyperglycemic state (FIG. 8). The derivative (40) also demonstrated a dose-dependent increase in PEPCK expression but was slightly less active than the parent compound (24). While both 24 and 40 had approximately equal efficacy to rosiglitazone when all three compounds were administered at 10 PM, the 5 and 1 μM doses of 24 and 40 were not effective.

### PPAR-γ Bioactivity of 24 and 40 in Diabetic Zebrafish

According to the PPAR-γ Western blot results, 24 is an excellent antidiabetic lead, exhibiting greater PPAR-γ expression than the FDA-approved PPAR-γ agonist when administered at the same concentration (10 IM). Both 24 and 40 demonstrated dose-dependent activation of PPAR-γ, but 40 was not as effective as 24.

### NFκB Bioactivity of 24 and 40 in Diabetic Zebrafish

The Western blot results for NFκB were also unexpected. The positive control did not significantly inhibit NFκB expression. In addition, compound 24 exhibited an opposite dose-dependent effect and 40, while demonstrating less NFκB expression, did not appear dose-dependent. It is likely that 40 was not administered at a high enough dose (or soluble enough) for an observable effect, but additional experiments are necessary to understand the NFκB results of the controls and 24. Although the NFκB expression of 40 was not strictly dose-dependent, it did significantly inhibit NFκB expression versus the untreated, diabetic control (more so than the rosiglitazone positive control group), despite solubility challenges. Additionally, 40 inhibited NFκB translocation (10.2 μM) in the in vitro assay. Having demonstrated NFκB inhibition in two different ways of analysis and both in vitro and in vivo, compound 40 seems to be an effective NFκB inhibitor worthy of further investigation. A reasonable explanation for the opposite dose-dependent effect observed in the NFκB expression for 24 is that NFκB activation occurred, peaking immediately before transcriptional inhibition. This hypothesis may be confirmed by a kinetic study in vivo, which may also help to resolve greater NFκB inhibition in rosiglitazone treated diabetic zebrafish by determining the peak inhibitory response time.

### CONCLUSIONS

A library from natural sources was obtained that consisted of pure natural product compounds that were known to exhibit some antidiabetic properties, particularly hypoglycemic effects. Following a brief investigation of their potential antidiabetic effects through the in vitro panel of assays, the diverse library of natural product compounds was used to validate the larval diabetic zebrafish assay developed herein.

A model of type 2 diabetes was established in larval zebrafish by absorption of the diabetogenic agent, alloxan, administered during an early developmental phase so that partial regeneration of pancreatic p-cells provided relative insulin deficiency. Although analysis of glucose levels proved to be difficult in such a small animal, the small size of larval zebrafish provided the ability to test numerous samples in a single experiment, in vivo investigation of test agents that are too small for more common animal study requirements, and ease of handling in comparison to murine and other animal models.

Diabetogenesis and treatment with potential antidiabetic compounds were achieved in a four-phase treatment cycle of incubation in solutions of alloxan (3 hours); 10 μM or 20 μg/mL of the treatment agent for compounds or extracts, respectively (3 hours); 30 minutes of glucose exposure (10 mg/mL); and one hour rinse in fish water. Since blood-glucose analysis was not successfully achieved via glucometer, Amplex Red, or a clinical HbA1C analyzer, molecular targets and metabolic enzymes were studied through Western blot analysis.

Evaluation of the diverse library of natural products through the PPAR-γ in vitro assay and the larval diabetic zebrafish Western blot assay, led to the identification of three structurally similar 4-phenylcoumarin glycosides (22-24) that exhibited significant hypoglycemic effects. Of the three compounds, 24 was selected for its superior performance in HbA1C and PPAR-γ expression levels. Thus 24 was derivatized via etherification to yield a new compound, the isopropyl ether derivative 40. While the derivative was not as effective as 24 in PPAR-γ activity, it did demonstrate effective NFκB inhibition both in vitro and in vivo, and so could be described as a novel natural product derivative and potential anti-inflammatory agent.

Preliminary docking analysis of a derivative
compared to parent compound

| | | Estimated | | | |
|---|---|---|---|---|---|
| | | free energy | Estimated | | |
| | | of binding | inhibition | | Binding Pocket |
| Compound | Protein | (kcal/mol) | constant | | Amino Acids |
| Der- | PPAR-G | −12.31 | 953.18 | pM | Phe264, Glu343, |
| 15R1P3 | | | | | Leu228, Glu295 |
| Der- | NFkB- | −5.44 | 109.88 | uM | Asp239, Ala239, |
| 15R1P3 | p50 | | | | Asn103, Val212 |
| Der- | NFkB- | −7.55 | 2.94 | uM | Phe184, His181, |
| 15R1P3 | p65 | | | | Glu49, Arg278 |
| Parent | PPAR-G | −11.97 | 1.69 | nM | Ile341, Gly284, |
| | | | | | Glu222, Lys265 |

These derivatives were also screened in silico against other target(s) (PARP-5/PEPCK) and data collected show their potential against these targets.

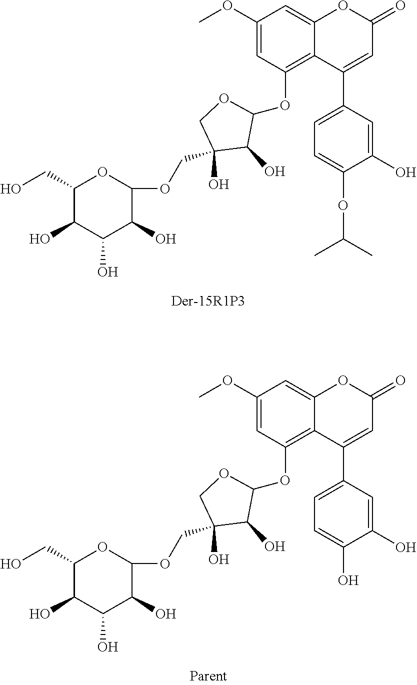 [SMILES: COc1cc(OC2OC[C@@](O)(COC3O[C@@H](CO)[C@H](O)[C@@H](O)[C@@H]3O)[C@@H]2O)c2c(-c3ccc(OC(C)C)c(O)c3)cc(=O)oc2c1 ; COc1cc(OC2OC[C@@](O)(COC3O[C@@H](CO)[C@H](O)[C@@H](O)[C@@H]3O)[C@@H]2O)c2c(-c3ccc(O)c(O)c3)cc(=O)oc2c1]

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound of Formula I: 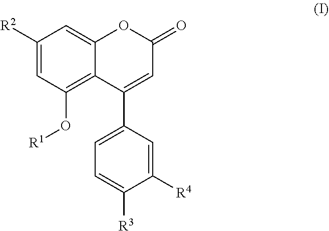
or a pharmaceutically acceptable salt, prodrug, or derivative thereof,
wherein:
R¹ is 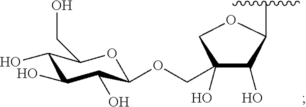
R², R³, and R⁴ are each independently selected from hydrogen, halo, cyano, azido, C₁-C₆alkyl, C₁-C₆haloalkyl, R^{x}O-, and R^{x}R^{y}N-, wherein R³ and R⁴ cannot both hydroxyl;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

2. The compound of claim 1, wherein R² is selected from halogen, C₁-C₆ alkyl, and R^{x}O-, wherein R^{x} is C₁-C₆ alkyl.

3. (canceled)

4. The compound of claim 1, wherein R² is R^{x}O-, wherein R^{x} is selected from methyl, ethyl, n-propyl, and isopropyl.

5. The compound of claim 1, wherein R² is methoxy.

6. The compound of claim 1, wherein R³ is selected from halogen, C₁-C₆ alkyl, and R^{x}O-, wherein R^{x} is hydrogen or C₁-C₆ alkyl.

7. (canceled)

8. The compound of claim 1, wherein R⁴ is selected from halogen, C₁-C₆ alkyl, and R^{x}O-, wherein R^{x} is hydrogen or C₁-C₆ alkyl.

9. (canceled)

10. The compound of claim 1, wherein R³ is C₁-C₆ alkoxy and R⁴ is hydroxy, or
wherein R³ is hydroxy and R⁴ is C₁-C₆ alkoxy, or
wherein R³ and R⁴ are each C₁-C₆ alkoxy.

11. The compound of claim 10, wherein R³ is isopropoxy, or
wherein R⁴ is isopropoxy, or
wherein R³ and R⁴ are each isopropoxy.

12-15. (canceled)

16. A pharmaceutical composition comprising a compound of claim 1, or a pharmaceutically acceptable salt, prodrug, or derivative thereof, and a pharmaceutically acceptable carrier or excipient.

17. A method for treating inflammation or for diminishing or ameliorating one or more symptoms caused by inflammation in a subject in need thereof comprising administering a compound of claim 1, or a pharmaceutically acceptable salt, prodrug, or derivative thereof.

18-19. (canceled)

20. The method of claim 17, wherein the inflammation is acute inflammation or chronic inflammation.

21. (canceled)

22. The method of claim 17, wherein the inflammation is associated with an inflammatory disorder, arthritis, or a gastrointestinal condition.

23-29. (canceled)

30. A method for the treatment of a systemic inflammatory disorder or for ameliorating or diminishing one or more inflammatory symptoms of a systemic inflammatory disorder in a subject in need thereof comprising administering a compound of claim 1, or a pharmaceutically acceptable salt, prodrug.

31. (canceled)

32. The method of claim 30, wherein the systemic inflammatory disorder is selected from non-alcoholic fatty liver disease, non-alcoholic steatohepatitis, inflammatory bowel disease, Crohn's disease, ulcerative colitis, psoriasis, irritable bowel syndrome, ankylosing spondylitis, osteoporosis, rheumatoid arthritis, psoriatic arthritis, chronic obstructive pulmonary disease, atherosclerosis, pulmonary arterial hypertension, pyridoxine-dependent epilepsy, atopic dermatitis, rosacea, multiple sclerosis, systemic lupus erythematosus, lupus nephritis, sepsis, eosinophilic esophagitis, chronic kidney disease, fibrotic renal disease, chronic eosinophilic pneumonia, extrinsic allergic alveolitis, pre-eclampsia, endometriosis, polycystic ovary syndrome, and cyclophosphamide-induced hemorrhagic cystitis.

33. A method for treating inflammation or for diminishing or ameliorating one or more symptoms caused by inflammation in a subject in need thereof comprising administering a therapeutically effective amount of a compound of Formula II 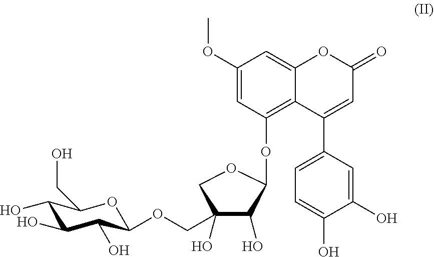
or a pharmaceutically acceptable salt, prodrug, or derivative thereof.

34-35. (canceled)

36. The method of claim 33, wherein the inflammation is acute inflammation or chronic inflammation.

37. (canceled)

38. The method of claim 33, wherein the inflammation is associated with an inflammatory disorder, arthritis, or a gastrointestinal condition.

39-45. (canceled)

46. A method for the treatment or for ameliorating or diminishing one or more inflammatory symptoms of a systemic inflammatory disorder in a subject in need thereof comprising administering a therapeutically effective amount of a compound of Formula II 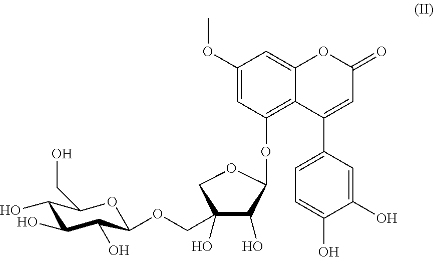
or a pharmaceutically acceptable salt, prodrug, or derivative thereof.

47. (canceled)

48. The method of claim 46, wherein the systemic inflammatory disorder is selected from non-alcoholic fatty liver disease, non-alcoholic steatohepatitis, inflammatory bowel disease, Crohn's disease, ulcerative colitis, psoriasis, irritable bowel syndrome, ankylosing spondylitis, osteoporosis, rheumatoid arthritis, psoriatic arthritis, chronic obstructive pulmonary disease, atherosclerosis, pulmonary arterial hypertension, pyridoxine-dependent epilepsy, atopic dermatitis, rosacea, multiple sclerosis, systemic lupus erythematosus, lupus nephritis, sepsis, eosinophilic esophagitis, chronic kidney disease, fibrotic renal disease, chronic eosinophilic pneumonia, extrinsic allergic alveolitis, pre-eclampsia, endometriosis, polycystic ovary syndrome, and cyclophosphamide-induced hemorrhagic cystitis.
